(19) United States
(12) Patent Application Publication
(10) Pub. No.: US 2024/0122933 A1
(43) Pub. Date: Apr. 18, 2024

(54) USE OF ISOQUINOLINE-1,5-DIAMINES FOR WOUND HEALING

(71) Applicant: Lutris Pharma Ltd., Tel Aviv (IL)

(72) Inventor: Noa Shelach, Tel Aviv (IL)

(21) Appl. No.: 18/534,307

(22) Filed: Dec. 8, 2023

Related U.S. Application Data
(63) Continuation of application No. PCT/IB2022/000384, filed on Jun. 30, 2022.
(60) Provisional application No. 63/217,862, filed on Jul. 2, 2021.

(51) Int. Cl. A61K 31/52; A61K 9/06; A61K 38/18; A61K 47/10; A61K 47/14; A61K 47/24; A61K 47/44; A61P 17/02

(52) U.S. Cl. CPC A61K 31/52; A61K 9/06; A61K 38/1858; A61K 47/10; A61K 47/14; A61K 47/24; A61K 47/44; A61P 17/02

(57) ABSTRACT

Compositions comprising an isoquinoline-1,5-diamine BRaf inhibitor and optionally an additional therapeutic agent are provided, also disclosed are methods of treatment, healing, and/or amelioration of wounds by topical administration of a therapeutically effective amount of the composition of this invention to a wound in a subject in need thereof.

### RELATED APPLICATIONS

This application claims priority to and benefit of U.S. Provisional Patent Application Ser. No. 63/217,862, filed on Jul. 2, 2021, the entire content of which is hereby incorporated by reference in its entirety

### BACKGROUND OF THE INVENTION

Wound healing is a complex process characterized by dynamic changes of the wound microenvironment to recruit and direct the different participating cell types. The entire process is classically divided in four consecutive and overlapping phases: hemostasis, inflammation, proliferation, and remodeling.

### SUMMARY OF THE INVENTION

Compositions comprising an isoquinoline-1,5-diamine BRaf inhibitor exhibiting a strong paradoxical Mitogen-Activated Protein Kinase (MAPK) increase (instead of the expected inhibition) and optionally an additional active agent are provided.

Also disclosed is a method of healing, treatment and/or amelioration of wounds by administrating a therapeutically effective amount of the composition of the disclosure to a subject in need thereof.

In some embodiments, the pharmaceutical composition comprises a therapeutically effective amount of a compound of Formula (I) or combinations thereof, optionally a therapeutically effective amount of an additional therapeutic agent, and a pharmaceutically acceptable carrier or excipient,

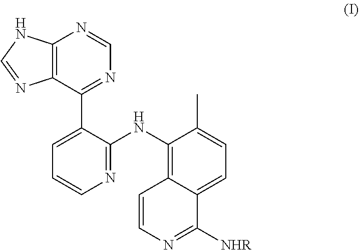

wherein R is 3-chloro-4-fluorophenyl, 2-fluoro-4-iodophenyl, 4-chloro-3-(trifluoromethyl)phenyl, 3-(1,1-dimethylethyl)-1-methyl-1H-pyrazol-5-yl, 3-(trifluoromethoxy)phenyl, or a pharmaceutical acceptable salt or solvate thereof, wherein the optional additional therapeutic agent comprises a pro-angiogenic agent, a growth factor, a sugar, an antacid, vitamin A, vitamin D, an antimicrobial, an antiseptic, an analgesic, a matrix metalloproteinase, a delta-like ligand 4, class 3 Semaphorin or combinations thereof.

In some embodiments, the compound of Formula (I), R is 4-chloro-3-(trifluoromethyl)phenyl.

In some embodiments, the compound of Formula (I), R is 3-(trifluoromethoxy)phenyl.

In some embodiments, the compound of Formula (I), R is 3-(1,1-dimethylethyl)-1-methyl-1H-pyrazol-5-yl.

In some embodiments, the additional therapeutic agent is a pro-angiogenic agent, a growth factor, a sugar, an antacid, vitamin A, vitamin D, an antimicrobial, an antiseptic, an analgesic or a combination thereof. In some embodiments, the growth factor comprises is a fibroblast growth factor, a vascular endothelial growth factor (VEGF), a platelet-derived growth factor (PDGF), a placental growth factor (PIGF), an angiopoietin, or a combination thereof.

In some embodiments, the additional therapeutic agent is a fibroblast growth factor, a vascular endothelial growth factor (VEGF), a platelet-derived growth factor (PDGF), a placental growth factor (PIGF), an angiopoietin, a matrix metalloproteinase (MMP), a delta-like ligand 4 (Dll4), a class 3 Semaphorin (SEMA3) or a combination thereof.

In some embodiments, the additional therapeutic agent is becaplermin or aCT1 peptide.

In some embodiments, the composition is formulated for topical administration.

In some embodiments, the composition is in the form of a gel, a hydrogel, an ointment, a cream, a foam, a spray, a lotion, a liquid or a dermal patch. In some embodiments, the compound of Formula (I) is present at a concentration of 0.01% w/w to 2% w/w of the total weight of the composition.

In some embodiments, the compound of Formula (I) is present at a concentration of 2% w/w to 5% w/w of the total weight of the composition.

Aspects of the disclosure relate to a method of treatment, healing and/or ameliorating a wound in a subject in need thereof, comprising administering the pharmaceutical composition comprising the therapeutically effective amount of the compound of Formula (I) and optionally a therapeutically effective amount of an additional therapeutic agent, wherein a sufficient amount of the pharmaceutical composition increases activity of Mitogen-Activated Protein Kinase by at least 2 times compared to activity of Mitogen-Activated Protein Kinase of a composition comprising a compound of Formula (I) wherein R is p-chlorophenyl, and wherein a sufficient amount of the composition is effective for treatment, healing, and/or amelioration of a wound.

Aspects of the disclosure relate to the use of pharmaceutical composition comprising a therapeutically effective amount of a compound of Formula (I) or combinations thereof, optionally a therapeutically effective amount of an additional therapeutic agent, and a pharmaceutically acceptable carrier or excipient, wherein R is 3-chloro-4-fluorophenyl, 2-fluoro-4-iodophenyl, 4-chloro-3-(trifluoromethyl)phenyl, 3-(1,1-dimethylethyl)-1-methyl-1H-pyrazol-5-yl, 3-(trifluoromethoxy)phenyl, or a pharmaceutical acceptable salt or solvate thereof, wherein a sufficient amount of the pharmaceutical composition increases activity of Mitogen-Activated Protein Kinase by at least 2 times compared to activity of Mitogen-Activated Protein Kinase of a composition comprising a compound of Formula (I) wherein R is p-chlorophenyl, and wherein a sufficient amount of the pharmaceutical composition is effective for treatment, healing, and/or amelioration of a wound.

In some embodiments, R is 4-chloro-3-(trifluoromethyl)phenyl.

In some embodiments, R is 3-(trifluoromethoxy)phenyl.

In some embodiments, R is 3-(1,1-dimethylethyl)-1-methyl-1H-pyrazol-5-yl.

In some embodiments, the wound is a non-penetrating wound selected from abrasions, lacerations and contusions, a penetrating wound selected from stab wounds, superficial cuts, scratches or lacerations, post-operative wounds, surgical incisions and wounds and gunshot wounds, a thermal wound selected from burns, sunburns and frostbite, an ulcer selected from chronic diabetic ulcers and pressure ulcers/bedsores, a chemical wound, animal or insect bites and stings and electrical wounds.

In some embodiments, the wound is caused by a disorder, and wherein the disorder is Epidermolysis Bullosa (EB), Stevens-Johnson Syndrome (SJS), Toxic Epidermal Necrolysis (TEN), Staphylococcal Scaled Skin Syndrome (SSSS). Pemphigus vulgaris (PV), or Toxic Shock Syndrome (TSS).

In some embodiments, the pharmaceutical composition is administered topically.

In some embodiments, the pharmaceutical composition is administered topically in the form of a gel, a hydrogel, an ointment, a cream, a spray, a wound dressing, a dermal patch, a foam, a lotion or a liquid.

Aspects of the disclosure relate to the use of a compound of Formula (I) or combinations thereof for the preparation of a medicament for the treatment, healing and/or ameliorating a wound in a subject in need thereof, wherein R is 3-chloro-4-fluorophenyl, 2-fluoro-4-iodophenyl, 4-chloro-3-(trifluoromethyl)phenyl, 3-(1,1-dimethylethyl)-1-methyl-1H-pyrazol-5-yl, 3-(trifluoromethoxy)phenyl, or a pharmaceutical acceptable salt or solvate thereof, and optionally an additional therapeutic agent.

### DETAILED DESCRIPTION OF THE INVENTION

Various embodiments are described hereinafter. It should be noted that the specific embodiments are not intended as an exhaustive description or as a limitation to the broader aspects discussed herein. One aspect described in conjunction with a particular embodiment is not necessarily limited to that embodiment and can be practiced with any other embodiment(s).

Unless defined otherwise, all technical and scientific terms used herein have the same meaning as commonly understood to one of ordinary skill in the art to which this invention pertains. Otherwise, certain terms used herein have the meanings as set forth in the specification. All patents, published patent applications and publications cited herein are incorporated by reference as if set forth fully herein. It must be noted that as used herein and in the appended claims, the singular forms “a,” “an,” and “the” include plural references unless the context clearly dictates otherwise.

The term “about” when used before a numerical designation, e.g., temperature, time, amount, and concentration, including range, indicates approximations which may vary by (+) or (−) 10%, 5% or 1%.

The term “pharmaceutically acceptable” as used herein refers to ingredients, agents, or compositions that are suitable for pharmaceutical administration without undue toxicity, incompatibility, instability, irritation, allergic response and the like.

The terms “pharmaceutically acceptable salts” or “salts thereof” mean salts which are pharmaceutically acceptable, and which possess the desired pharmacological activity.

The term “carrier” as used herein, refers to nontoxic chemical compounds or agents that facilitate the incorporation of a compound into cells, e.g., dermal cells, or tissues. Carriers useful herein include any such materials known in the art, which are nontoxic and do not interact with other components of the formulation in which it is contained in a deleterious manner. As used herein, “pharmaceutically acceptable carrier” includes any and all solvents, dispersion media, and the like, which are pharmaceutically acceptable. Examples of pharmaceutically acceptable carriers include but are not limited to sugars, starches, cellulose, excipients, oils, glycols, polyols, esters, agar, and buffering agents. The above are non-limiting examples of carriers.

The term “therapeutically effective amount” of a compound or a composition refers to that amount of the compound or the composition that results in treatment, including reduction or inhibition of symptoms in a patient.

The term “subject” as used herein refers to organisms to be treated by the compounds or compositions of the present disclosure. Such organisms include a mammal, including a human or non-human mammal. The terms “patient”, “individual” and “subject” may be used interchangeably.

A number of novel BRaf inhibitors of Formula (I) have been disclosed in U.S. Patent Application Publication No. U.S.20190389862 and their use for the treatment of cutaneous reactions induced by chemotherapy agents such as EGFR inhibitors, PI3K inhibitors, MEK inhibitors or combinations thereof has been reported.

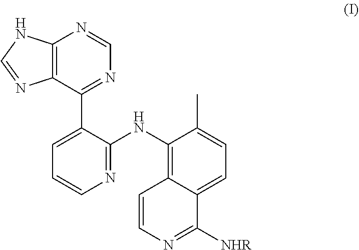

BRaf is a signal transduction protein kinase involved in the regulation of the mitogen activated protein kinase (MAPK or ERK) signaling pathway.

Shelach N. showed in the U.S. Patent Application Publication No. U.S.20180369247 that Formula (I) compounds exhibiting paradoxical MAPK increase (instead of the expected inhibition) can be put to good use for treating cutaneous reactions side-effects caused by treatment with EGFR or P13K inhibitors.

Out of the BRaf inhibitors investigated in U.S. Patent Application Publication No. U.S.20190389862, the compound of Formula (I) wherein R=3-(trifluoromethoxy)phenyl (LUT-014) was selected as clinical studies candidate for treatment of cutaneous reactions such as acneiform rash.

BRaf compounds having structures different from the compounds of Formula (I) of this disclosure have been investigated for wound healing activity (Ribas A. et al., U.S. Patent Application Publication Nos. U.S.20170100345 and U.S.20190262343).

Wound healing is a complex process characterized by dynamic changes of the wound microenvironment to recruit and direct the different participating cell types. The entire process is classically divided in four consecutive and overlapping phases: hemostasis, inflammation, proliferation, and remodeling. (E. Öhnstedt et al. (2019) “The discovery and development of topical medicines for wound healing”, Expert Opinion on Drug Discovery, 14:5, 485-497″).

Though many wound dressings are available, few registered pharmaceutical options are today available to accelerate healing for wound healing as active medical products by the Food and Drug Administration (FDA). One of the FDA-approved products is becaplermin (Regranex™). Becaplermin is FDA-approved for promoting healing of diabetic foot ulcers.

There is a need in the art for new drugs, methods of screening for the compounds in a population of BRaf candidates and new compositions effective in healing, treating and/or ameliorating wounds.

Aspects of the disclosure provide compositions comprising a therapeutically effective amount of a compound of Formula (I) or a pharmaceutically acceptable salt or a solvate thereof, optionally an additional therapeutic agent, and a pharmaceutically acceptable topical carrier or excipient, wherein a sufficient amount of the composition increases activity of Mitogen-Activated Protein Kinase by at least 2 times compared to activity of Mitogen-Activated Protein Kinase of a composition comprising a compound of Formula (I), wherein R is p-chlorophenyl and wherein a sufficient amount of the composition is effective for healing, treating and/or ameliorating a wound.

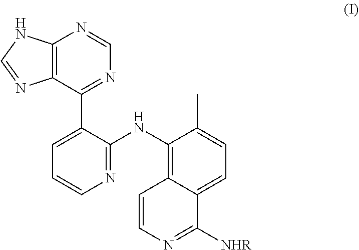

The R substituent in the compounds of Formula (I) is 3-chloro-4-fluorophenyl, 2-fluoro-4-iodophenyl, 4-chloro-3-(trifluoromethyl)phenyl, 3-(1,1-dimethylethyl)-1-methyl-1H-pyrazol-5-yl, 3-(trifluoromethoxy)phenyl or a combination thereof.

The above compounds of Formula (I) are isoquinoline-1,5-diamine BRaf inhibitors first disclosed by Shelach N. in U.S. Patent Application Publication No. U.S.20190389862, which reports their use for the treatment of cutaneous reactions induced by chemotherapy agents such as EGFR inhibitors, PI3K inhibitors, MEK inhibitors or combinations thereof.

The present embodiment discloses a new therapeutic use, namely wound healing, for compositions comprising the above isoquinoline-1,5-diamine BRaf inhibitors exhibiting a strong paradoxical Mitogen-Activated Protein Kinase (MAPK) increase instead of the expected inhibition.

BRaf is a signal transduction protein kinase involved in the regulation of the Mitogen Activated Protein Kinase (MAPK or ERK) signaling pathway. Mutations in BRaf can induce constitutive signaling through the MAPK pathway which may result in uncontrolled cell proliferation. The use of BRaf inhibitors has been demonstrated to be associated with inhibition of MAPK signaling, as can be determined by inhibition or reduction in level of phosphorylated ERK, which is the downstream effector of BRaf. Yet, some BRaf inhibitors can paradoxically induce an opposite effect of activation of MAPK signaling in BRaf wild-type cells (as determined by increased levels of phosphorylated ERK). The underlying mechanisms of paradoxical MAPK activation have been attributed to promotion of wild-type BRAF and CRAF dimerization and transactivation of the noninhibited RAF protein leading to subsequent MAPK pathway activation.

A series of orally bioavailable kinase inhibitors disclosed by Smith A.L. et al. (J. Med. Chem. 2009, 52, 6189-6192) showed potent biochemical activity. Compound 1 of the series (C-1) showed the greatest potency (^{WT}B-Raf Ki=1 nmol/L, V600EB-Raf Ki=1 nmol/L, and C-Raf Ki=0.3 nmol/L) and was therefore used in this disclosure as reference compound.

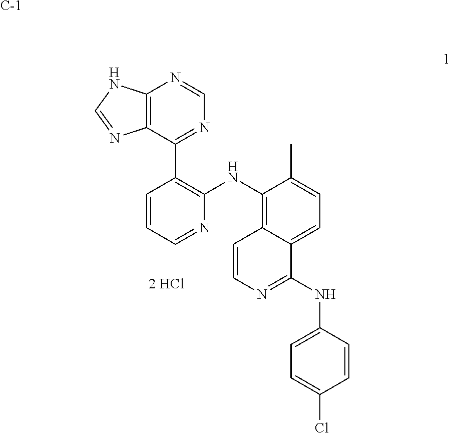

Carnahan J. et al. (Mol. Cancer Ther. 9(8) August 2010) found that in cells with wild-type BRaf and mutated KRAS, exposure to Raf inhibitors resulted in a dose-dependent and sustained paradoxical effect of activation of Mitogen-Activated Protein Kinase signaling. Raf inhibition led to entry into the cell cycle, enhanced proliferation.

This disclosure reports the screening for use in wound healing out of the BRaf inhibitors investigated in U.S. Patent Application U.S.20190389862, five BRaf inhibitors exhibiting an increase in the activity of Mitogen-Activated Protein Kinase (MAPK) by at least 2 times (see Table 1) compared to activity of Mitogen-Activated Protein Kinase of a compound of Formula (I) wherein R is p-chlorophenyl (reference compound C-1, disclosed in Smith A.L. et al., J. Med. Chem. 2009, 52, 6189-6192).

A population of BRaf inhibitors of Formula (I) (see Table 1) has been screened for MAPK activity, which was compared to the MAPK activity of the reference compound C-1 of Formula (I) wherein the R substituent is p-chlorophenyl. The compounds of Formula (I) in Table 1, code names from LUT-012 to LUT-20 exhibiting an MAPK activity increase (five out of nine Formula (I) compounds) have been screened out of the BRaf population and next the compounds showing the strongest MAPK activity increase (or highest LUT compound/C-1 MAPK ratio), namely three compounds LUT-013 (R=3-(1,1-dimethylethyl)-1-methyl-1H-pyrazol-5-yl, LUT-014 (R=3-(trifluoromethoxy)phenyl) and LUT-017 (R=4-chloro-3-(trifluoromethyl)phenyl), have been found to exhibit the highest LUT/C-1 MAPK ratio (between 2.9 and 3.5-fold) and strongest MAPK activity increase.

TABLE 1
MAPK Ratios of Compounds of Formula (I) vs. C-1

| | | FIGURE | LUT/C1 |
|---|---|---|---|
| | LUT | No. of appl. | MAPK |
| R Substituent | Code | US20199389862 | Ratios |
| 3-Ethynylphenyl | LUT-012 | 2A & 2B, 3A, 3C | 0.7 |
| 3-Chloro-4-fluorophenyl | LUT-015 | 3A, 3C | 2.1 |
| 2-Fluoro-4-iodo-phenyl | LUT-016 | 2A & 2B, 3A & 3C | 2.0 |
| 4-Chloro-3- | LUT-017 | 1A & 1B, 3A & | 3.5 |
| (trifluoromethyl)phenyl | | 3C, 4A, 4B | |
| 3-(1,1-Dimethylethyl)-1-methyl- | LUT-013 | 3A & 3C | 3.4 |
| 1H-pyrazol-5-y1 | | | |
| 3-(Trifluoromethoxy)phenyl | LUT-014 | 1A & 1B, 3A & 3C, | 2.9 |
| | | 4A, 4B | |
| 3.5-Dihydroxyphenyl | LUT-019 | | No |
| | | | activity |
| Phenyl-3-sulfonamide | LUT-020 | 3A & 3C | 1.0 |
| 3-(Trifluoromethyl)phenyl | LUT-018 | | No |
| | | | activity |
| p-Chlorophenyl (reference | C-1 | 2A & 2B, 3A & | 1.0 |
| compound) | | 3C, 4A, 4B | |

As shown in Table 1, the R substitution has a profound effect on the LUT/C-1 MAPK ratios

In some embodiments, the isoquinoline-1,5-diamine BRaf compounds, exhibit an unexpected high paradoxical MAPK increase. These isoquinoline-1,5-diamine BRaf compounds may be good candidates for other biological activities, such as wound healing activity.

The highest MAPK ratios were found for the compounds of Formula 1 LUT-013, LUT-014 and LUT-017, which were selected as lead compounds for testing their wound healing activity.

The BRaf inhibitors of this disclosure were screened for phosphorylation of ERK (MAPK).

The over-proliferation effects are consistent with the paradoxical effect that is demonstrated by phosphorylation of ERK (MAPK).

The most effective compounds of Formula (I) (those which induced highest MAPK/ERK activation), generate more dramatic over-proliferation, which can be beneficial for wound treatment.

Without wishing to be bound by theory, it is hypothesized by the inventor that the compounds of Formula (I) exhibiting the strongest LUT/C-1 MAPK ratio (strongest MAPK increase) are the best candidates for wound healing activity.

BRaf inhibitors in general are known to exhibit photo-toxicity, which is exclusively triggered by UVA radiation and resolves rapidly post-treatment discontinuation.

As the isoquinoline-1,5-diamine compounds of Formula (I) of this disclosure are intended mainly for wound healing by topical use, low phototoxicity is a pre-requisite.

The level of phototoxicity was determined by measuring a Photo-Irritation Factor (PIF) or a Mean Photo Effect (MPE) of one of the compounds of Formula (I), namely LUT-014 (R=3-(trifluoromethoxy)phenyl).

In one embodiment, one of the compounds of Formula (I) (LUT-014) of present disclosure exhibits low phototoxicity (exhibits a PIF of less than 2).

### Compositions

In some embodiments, provided herein is a pharmaceutical composition comprising a compound of Formula (I) a pharmaceutically acceptable salt or a solvate thereof or a combination thereof and a pharmaceutically acceptable carrier or excipient.

In some embodiments, provided herein is a pharmaceutical composition comprising a compound of Formula (I) a pharmaceutically acceptable salt or a solvate thereof, or a combination thereof, optionally an additional therapeutical agent, and a pharmaceutically acceptable carrier or excipient.

In some embodiments, the above compositions are formulated for topical administration.

In some embodiments, in the compound of Formula (I) R is 3-chloro-4-fluorophenyl, 2-fluoro-4-iodophenyl, 4-chloro-3-(trifluoromethyl)phenyl, 3-(1,1-dimethylethyl)-1-methyl-1H-pyrazol-5-yl, 3-(trifluoromethoxy)phenyl or a combination thereof.

In some embodiments, in the compound of Formula (I), R is 4-chloro-3-(trifluoromethyl)phenyl.

In some embodiments, in the compound of Formula (I), R is 3-(trifluoromethoxy)phenyl.

In some embodiments, in the compound of Formula (I), R is 3-(1,1-dimethylethyl)-1-methyl-1H-pyrazol-5-yl.

The compositions of the present disclosure may be used in the form of a topical pharmaceutical composition.

In some embodiments, the composition is administered topically in the form of a gel, a hydrogel, an ointment, a cream, a spray, a wound dressing, a dermal patch, a foam, a lotion or a liquid.

In some embodiments, the compositions described herein can comprise one or more of the following: one or more solvent (e.g. water, proteic solvent Transcutol P™), an hydrophobic excipient (e.g. white petrolatum, Isopropyl Palmitate), an emulsifier (e.g. Crodafos™ CES), a surfactant (e.g. Hexylene glycol), a preservative (e.g. parabens). Compositions formulated as a cream may contain a cream stabilizer, such as xanthan gum; an emulsifier, for example a non-ionic emulsifier; at least one liquid and one solid hydrophobic material such as liquid and solid fatty acids, fatty alcohols, fatty acid esters, pharmaceutical grades of waxes and hydrocarbons; a preservative; an antioxidant; and water. Suitable non-ionic emulsifiers are the polyoxyalkylene fatty acid esters, in particular the polyoxyalkylene stearates, such as polyoxyethylene 25 oxypropylenestearate, polyoxyl stearate and polyethylene glycol 400 monostearate. The hydrophobic material may be any pharmaceutically acceptable fatty material known and used by those skilled in the art. Such hydrophobic materials include the fatty acids, fatty alcohols and fatty acid esters, wherein the fatty acid moiety has from about 12 to about 20 carbon atoms, such as stearyl alcohol, stearic acid, isopropyl myristate and cetyl alcohol; as well as pharmaceutical grades of beeswax, including white wax, sperm wax, lanolin, mineral oil, etc.

In some embodiments, the optional additional therapeutic agent is selected from the group consisting of a pro-angiogenic agent, a growth factor, a sugar, an antacid, vitamin A, vitamin D, an antimicrobial, an antiseptic and an analgesic. The optional therapeutic agent can be a natural or synthetic analog or derivatives of vitamin A, vitamin D or a sugar. For example, the vitamin A derivative can be retinoic acid.

In some embodiments, the above optional additional therapeutic agent growth factor is a fibroblast growth factor, a vascular endothelial growth factor (VEGF), a platelet-derived growth factor (PDGF), a placental growth factor (PIGF), an angiopoietin, a matrix metalloproteinase (MMP), a delta-like ligand 4 (Dll4), a class 3 Semaphorin (SEMA3) or a combination thereof. In some embodiments, the growth factors are recombinant growth factors.

In some embodiments, the above optional therapeutic pro-angiogenic agent is a syndecan-4 proteoliposome, a short, antimicrobial peptide (AMP, e.g. a cathelicidin).

In some embodiments, the above optional therapeutic sugar is honey or white sugar.

In some embodiments, the above optional additional antacid therapeutic agent is sucralfate or Tums (for duodenal ulcers).

In some embodiments, the above optional additional antimicrobial therapeutic agent is silver, iodine, potassium permanganate, chlorhexidine or polyhexamethyl guanidine (PHMG).

In some embodiments, the above optional additional analgesic therapeutic agent is ibuprofen or acetaminophen.

Exemplary compositions of the compounds of Formula (I) LUT-014 (R=3-(trifluoromethoxy)phenyl), LUT-017 (R=4-chloro-3-(trifluoromethyl)phenyl) and LUT-013 (R=3-(1,1-dimethylethyl)-1-methyl-1H-pyrazol-5-yl)) are provided (Examples 1, 2 and 7).

Also provided are exemplary compositions and methods of treatment using the compound of Formula (I) LUT-017 (R=4-chloro-3-(trifluoromethyl)phenyl) in combination with an additional therapeutic agent (becaplermin) (Examples 3, 5 and 6),

Becaplermin, sold in the U.S. as Regranex™, is a recombinant human platelet-derived growth factor (PGDF) FDA-approved for promoting healing of diabetic foot ulcers (DFU). Given alone, it has only a modest beneficial effect in promoting wound healing.

In some embodiments, the combination treatment provides a better healing effect, due to synergy.

Another additional therapeutic agent is the synthetic peptide aCT1 being developed for FDA approval by Firstring Research Inc. as Granexin gel. The aCT1 peptide acts at the inflammation and scar formation wound healing stages.

In some embodiments, the optional additional therapeutic agent is selected from becaplermin and aCT1 peptide.

In some embodiments, a combination treatment of the two or more therapeutic agents can lead to beneficial wound healing synergy. In some embodiments, the combination therapy comprises administering a compound of Formula (I) and an additional therapeutic agent is selected from becaplermin and aCT1 peptide. Without being bound to the theory, as the compounds of Formula (I) disclosed herein act mostly at the proliferation wound healing stage and the aCT1 peptide acts at the inflammation and scar formation wound healing stages, itis believed that a combination treatment of the two therapeutic agents will lead to beneficial wound healing synergy.

The combination treatment may be administered as a single combination topical composition comprising a compound of Formula (I) together with the additional therapeutic agent (Example 5), or as two separate topical compositions administered for example on alternate days or alternate weeks, the first composition comprising a compound of Formula (I) and the second composition comprising the additional therapeutic agent (Example 6).

Said compositions are used for the healing, treatment and/or amelioration of wounds.

In some embodiments, the compound of Formula (I) is present in the pharmaceutical composition of this disclosure at a concentration of 0.01% w/w to 2% w/w of the total weight of the composition.

In some embodiments, the compound of Formula (I) is present in the pharmaceutical composition of this disclosure at a concentration of 2% w/w to 5% w/w of the total weight of the composition.

In some embodiments, the pharmaceutical composition may comprise about 0.01% w/w to about 2% w/w of the compound of Formula (I) or a pharmaceutically acceptable salt or a solvate thereof, or a combination thereof, based on the total weight of the composition. In some embodiments, the pharmaceutical composition may comprise about 0.01% w/w to about 0.1% w/w of the compound or a pharmaceutically acceptable salt or a solvate thereof, or a combination thereof, based on the total weight of the composition. For example, the pharmaceutical composition may comprise about 0.01, 0.02, 0.03, 0.04, 0.05, 0.06, 0.07, 0.08, 0.09, 0.1% w/w, including values and ranges therebetween, of any of the compounds disclosed herein or combinations thereof. In some embodiments, the pharmaceutical composition may comprise from about 0.01% to about 0.03%, about 0.01% to about 0.04%, about 0.01% to about 0.05%, about 0.01% to about 0.06%, about 0.01% to 0.07%, about 0.01% to about 0.08%, about 0.01% to about 0.09%, about 0.01% to about 0.1%, 0.02% to about 0.03%, about 0.02% to about 0.04%, about 0.02% to about 0.05%, about 0.02% to about 0.06%, about 0.02% to 0.07%, about 0.02% to about 0.08%, about 0.02% to about 0.02%, about 0.02% to about 0.1%, 0.03% to about 0.04%, about 0.03% to about 0.05%, about 0.03% to about 0.06%, about 0.03% to 0.07%, about 0.03% to about 0.08%, about 0.03% to about 0.09%, about 0.03% to about 0.1%, about 0.04% to about 0.05%, about 0.04% to about 0.06%, about 0.04% to 0.07%, about 0.04% to about 0.08%, about 0.04% to about 0.09%, about 0.04% to about 0.1%, %, about 0.05% to about 0.06%, about 0.05% to 0.07%, about 0.05% to about 0.08%, about 0.05% to about 0.09%, about 0.05% to about 0.1% weight/weight (w/w), including values and ranges therebetween, of any of the compounds disclosed herein.

In some embodiments, the pharmaceutical composition may comprise about 0.03% w/w to about 0.25% w/w of the compound of Formula (I) or a pharmaceutically acceptable salt or a solvate thereof, or a combination thereof, based on the total weight of the composition. For example, the pharmaceutical composition may comprise about 0.03, 0.04, 0.05, 0.06, 0.07, 0.08, 0.09, 0.1, 0.11, 0.12, 0.13, 01.4, 0.15, 0.16, 0.17, 0.18, 0.19, 0.20, 0.21, 0.22, 0.23, 0.24, 0.25% w/w, including values and ranges therebetween, of any of the compounds disclosed herein or combinations thereof. In some embodiments, the pharmaceutical composition may comprise from about 0.03% to about 0.04%, about 0.03% to about 0.05%, about 0.03% to about 0.06%, about 0.03% to 0.07%, about 0.03% to about 0.08%, about 0.03% to about 0.09%, about 0.03% to about 0.1%, about 0.03% to about 0.11%, about 0.03% to about 0.12%, about 0.03% to about 0.13%, about 0.03% to about 0.14%, about 0.03% to about 0.15%, about 0.03% to about 0.16%, about 0.03% to about 0.17%, about 0.03% to about 0.18%, about 0.03% to about 0.19%, about 0.03% to about 0.2%, about 0.03% to about 0.21%, about 0.03% to about 0.22%, about 0.03% to about 0.23%, about 0.03% to about 0.24%, about 0.03% to about 0.25 weight/weight (w/w), including values and ranges therebetween, of any of the compounds disclosed herein.

In some embodiments, the pharmaceutical composition may comprise about 0.01% w/w to about 0.1% w/w of the compound of Formula (I) or a pharmaceutically acceptable salt or a solvate thereof, or a combination thereof, based on the total weight of the composition. For example, the pharmaceutical composition may comprise about 0.01, 0.02, 0.03, 0.04, 0.05, 0.06, 0.07, 0.08, 0.09, 0.1% w/w, including values and ranges therebetween, of any of the compounds disclosed herein or combinations thereof. In some embodiments, the pharmaceutical composition may comprise from about 0.01% to about 0.03%, about 0.01% to about 0.04%, about 0.01% to about 0.05%, about 0.01% to about 0.06%, about 0.01% to 0.07%, about 0.01% to about 0.08%, about 0.01% to about 0.09%, about 0.01% to about 0.1%, 0.02% to about 0.03%, about 0.02% to about 0.04%, about 0.02% to about 0.05%, about 0.02% to about 0.06%, about 0.02% to 0.07%, about 0.02% to about 0.08%, about 0.02% to about 0.02%, about 0.02% to about 0.1%, 0.03% to about 0.04%, about 0.03% to about 0.05%, about 0.03% to about 0.06%, about 0.03% to 0.07%, about 0.03% to about 0.08%, about 0.03% to about 0.09%, about 0.03% to about 0.1%, about 0.04% to about 0.05%, about 0.04% to about 0.06%, about 0.04% to 0.07%, about 0.04% to about 0.08%, about 0.04% to about 0.09%, about 0.04% to about 0.1%, %, about 0.05% to about 0.06%, about 0.05% to 0.07%, about 0.05% to about 0.08%, about 0.05% to about 0.09%, about 0.05% to about 0.1% w/w, including values and ranges therebetween, of any of the compounds disclosed herein.

In some embodiments, the pharmaceutical composition may comprise from about 0.25% to about 0.5%, about 0.5% to about 1.0%, about 1.0% to about 1.5%, about 1.5% to 2.0% weight/weight (w/w), including values and ranges therebetween, of any of the compounds disclosed herein of the compound of Formula (I) or a pharmaceutically acceptable salt or a solvate thereof.

In some embodiments, the pharmaceutical composition may comprise from about 0.01% to 25% of any of the compounds disclosed herein. For example, the pharmaceutical composition may comprise from about 0.01% to 25%, from about 0.01% to 20%, from about 0.01% to 15%, from about 0.01% to 10%, from about 0.01% to 5%, from about 0.01% to 1%, from about 0.03% to 25%, from about 0.03% to 20%, from about 0.03% to 10%, from about 0.03% to 15%, from about 0.03% to 10%, from about 0.03% to 5%, from about 0.03% to 1%, from about 0.1% to 25%, from about 0.1% to 20%, from about 0.1% to 15%, from about 0.1% to 10%, from about 0.1% to 5%, from about 0.1% to 1%, from about 1% to 25%, from about 1% to 20%, from about 1% to 15%, from about 1% to 10%, from about 1% to 5%, from about 2.0% to about 2.5%, about 2.5% to about 3.0%, about 3.0% to about 3.5%, about 3.5% to about 4.0%, about 4.0% to about 4.5%, about 4.5% to about 5.0% weight/weight (w/w), including values and ranges therebetween, of any of the compounds disclosed herein, of the compound of Formula (I) or a pharmaceutically acceptable salt or a solvate thereof.

In some embodiments, the pharmaceutical composition comprises from about 0.01% to 25% of the one or more additional agent. For example, the pharmaceutical composition may comprise 0.01% to 25%, 0.01% to 10%, 0.03% to 1%, 0.03% to 5%, or 1% to 10% w/v, or about 6%, 7% 8%, 9%, 10%, 15% or 20% w/v of the one or more additional agent.

### Method of Treatment

In an embodiment, there is provided a method of treatment, healing and/or ameliorating a wound in a subject in need thereof, comprising administering a pharmaceutical composition comprising a therapeutically effective amount of the compound of Formula (I) and optionally a therapeutically effective amount of an additional therapeutic agent, wherein a sufficient amount of the pharmaceutical composition increases activity of Mitogen-Activated Protein Kinase by at least 2 times compared to activity of Mitogen-Activated Protein Kinase of a composition comprising a compound of Formula (I) wherein R is p-chlorophenyl. and wherein a sufficient amount of the composition is effective for treatment, healing and/or ameliorating a wound.

In an embodiment, there is provided an use of a compound of Formula (I) and optionally an additional therapeutic agent for the preparation of a medicament for the treatment, healing and/or ameliorating a wound in a subject in need thereof.

In an embodiment, there is provided an use of a compound of Formula (I) and optionally an additional therapeutic agent for the treatment, healing and/or ameliorating a wound in a subject in need thereof, wherein a sufficient amount of the pharmaceutical composition increases activity of Mitogen-Activated Protein Kinase by at least 2 times compared to activity of Mitogen-Activated Protein Kinase of a composition comprising a compound of Formula (I) wherein R is p-chlorophenyl, and wherein a sufficient amount of the composition is effective for treatment, healing, and/or amelioration of a wound.

In some embodiments, the compound of Formula (I) or a pharmaceutical composition thereof as described above may be administered or delivered in combination with or in conjunction with one or more additional therapeutic agents. The compound of Formula (I) and the additional therapeutic agent(s) can act synergistically together.

In an embodiment, there is provided a method of treatment, healing and/or ameliorating a wound in a subject in need thereof, comprising administering a pharmaceutical composition comprising a synergistic therapeutically effective amount of the compound of Formula (I) and of an additional therapeutic agent, wherein a sufficient amount of the pharmaceutical composition increases activity of Mitogen-Activated Protein Kinase by at least 2 times compared to activity of Mitogen-Activated Protein Kinase of a composition comprising a compound of Formula (I) wherein R is p-chlorophenyl, and wherein a sufficient amount of the composition is effective for treatment, healing and/or ameliorating a wound.

In some embodiments, the composition can comprise one or more optional additional therapeutic agent. In some embodiments, the compound of Formula (I) can be administered simultaneously or sequentially with the additional therapeutic agent. In some embodiments, the compound of Formula (I) and one or more additional agent may be part of a single application or administration, or may be applied or administered separately. In some embodiments, the compound of Formula (I) and the additional agent can be administered at different times, for example several hours or several days apart.

The wound treated by the method of the disclosure can be, but is not limited to, a non-penetrating wound selected from abrasions, lacerations and contusions, a penetrating wound selected from stab wounds, superficial cuts, scratches or lacerations, post-operative wounds, surgical incisions and wounds and gunshot wounds, a thermal wound selected from burns, sunburns and frostbite, an ulcer selected from chronic diabetic ulcers and pressure ulcers/bedsores, a chemical wound, animal or insect bites and stings and electrical wounds.

Alternatively, the wound can be caused by a disorder. For example, the disorder can be Epidermolysis Bullosa (EB), Stevens-Johnson Syndrome (SJS), Toxic Epidermal Necrolysis (TEN), Staphylococcal Scaled Skin Syndrome (SSSS). Pemphigus vulgaris (PV), or Toxic Shock Syndrome (TSS).

The method of treatment, includes contacting the wound on a subject in need thereof with a therapeutically effective amount of the composition of the disclosure. For example, the composition of the disclosure can be applied directly to the wound.

Alternatively, the treatment may comprise the use of a wound dressing, wherein the composition is impregnated or coated on the dressing, and wherein the wound dressing is an alginate dressing, an antimicrobial dressing, a bandage, a Band-Aid, a biosynthetic dressing, a biological dressing, a collagen dressing, a composite dressing, a compression dressing, a contact layer dressing, a foam dressing, a gauze dressing, a hydrocolloid dressing, a hydrogel dressing, a skin sealant or liquid skin dressing, a specialty absorptive dressing, a transparent film dressing, or a wound filler. In some embodiments, the composition is allowed to stay in contact with the wound for a sufficient period of time to promote healing.

In some embodiments, the pharmaceutical composition of the disclosure comprising a compound of Formula (I) and optionally an additional therapeutic agent is impregnated or coated on one of the above wound dressings.

In some embodiments, once the composition is applied, a wound dressing may be applied over the topical composition(s). In some embodiments, the compound of Formula (I) and optionally an additional therapeutic agent may be administered using different dressings, treatment regimens, treatment modalities or a combination thereof.

In some embodiments, the topical pharmaceutical composition of the disclosure comprising a compound of Formula (I) and optionally an additional therapeutic agent is administered once daily for 1 week, 2 weeks, 3 weeks, 4 weeks, 5 weeks, 6 weeks, 8 weeks, etc., or until the wound is healed. In some embodiments, the topical composition is administered once daily for 4 to 8 weeks.

In some embodiments, the topical pharmaceutical composition comprising a compound of Formula (I) and optionally an additional therapeutic agent is administered twice daily for 1 week, 2 weeks, 3 weeks, 4 weeks, 5 weeks, 6 weeks, etc., or until the wound is healed. In some embodiments, the topical pharmaceutical composition is administered twice daily for 4 to 8 weeks.

In some embodiments, the topical pharmaceutical composition comprising a compound of Formula (I) and optionally an additional therapeutic agent is administered every other day for 1 week, 2 weeks, 3 weeks, 4 weeks, 5 weeks, 6 weeks, 8 weeks, etc., or until the wound is healed. In some embodiments, the topical composition is administered every other day for 4 to 8 weeks.

In some embodiments, the topical pharmaceutical composition comprising a compound of Formula (I) and optionally an additional therapeutic agent is administered every other day for 1 week, 2 weeks, 3 weeks, 4 weeks, 5 weeks, 6 weeks, 8 weeks, etc., or until the wound is healed. In some embodiments, the topical composition is administered twice a week for 4 to 8 weeks.

In some embodiments, the topical pharmaceutical composition comprising a compound of Formula (I) and optionally an additional therapeutic agent is administered once weekly for 1 week, 2 weeks, 3 weeks, 4 weeks, 5 weeks, 6 weeks, 8 weeks, etc., or until the wound is healed. In some embodiments, the topical composition is administered once weekly for 4 to 8 weeks.

An exemplary method of treatment using a compound of Formula (I) LUT-017 (R=4-chloro-3-(trifluoromethyl)phenyl) is provided (Example 4).

Also provided are exemplary methods of treatment using the compound of Formula (I) LUT-017 (R=4-chloro-3-(trifluoromethyl)phenyl) in combination with an additional therapeutic agent (becaplermin) (Examples 5 and 6),

The combination treatment may be administered as one topical composition comprising a compound of Formula (I) together with the additional therapeutic agent (Example 5). The combination treatment may be administered as two separate compositions administered for example on alternate days, the first composition comprising a compound of Formula (I) and the second composition comprising the additional therapeutic agent (Example 6). The combination treatment may be administered as two separate compositions administered for example on the same day, the first composition comprising a compound of Formula (I) and the second composition comprising the additional therapeutic agent.

The above methods of treatment are used similarly when the additional therapeutic agent in combination with a compound of Formula (I) is aCT1 peptide.

### EXAMPLES

The following examples illustrate certain embodiments of the invention but are not meant to limit the scope of the claims in any way. The following examples are put forth so as to provide those of ordinary skill in the art with a complete disclosure and description of how to make and use the described invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Centigrade, and pressure is at or near atmospheric.

### Synthesis and Biological Studies of the Compounds of Formula (I)

The chemical structures, synthetic schemes and the synthetic procedures for each of the compounds of Formula (I) of this disclosure, as well as their MAPK (ERK) and the phototoxicity studies of LUT-014 were detailed in Shelach N., U.S. Patent Application Publication No. U.S.2019389862, which is incorporated herein in its entirety.

The synthesis of the reference compound C-1 was detailed in Smith A.L. et al., J. Med. Chem. 2009, 52, 6189-6192.

### Example 1

LUT-017 (Formula (I), R=4-chloro-3-(trifluoromethyl)phenyl) 0.03% w/w Cream Composition

### Composition

### LUT-017 0.03% w/w

### White Petrolatum 10.0% w/w

### Isopropyl Palmitate 5.0% w/w

Cetearyl Alcohol (and) Dicetyl Phosphate (and) Ceteth-10 Phosphate (Crodafos™ CES) 10.0% w/w
Hexylene glycol 2.0% w/w
Diethylene glycol monoethyl ether (Transcutol PTM) 25.0% w/w

### Methylparaben 0.2% w/w

### Propylparaben 0.05% w/w

### Purified Water q.s. ad 100 (47.45%)

### Procedure

LUT-017 (0.03 grams), is weighed and charged into a 250 mL three-necked spherical glass flask provided with a mechanical stirrer, a thermometer and a funnel.

Diethylene glycol monoethyl ether (Transcutol P™, 25 g) is added gradually with stirring to the flask containing LUT-017, kept in a water bath heated at 50 deg C. Hexylene glycol (2 g) is then added gradually while stirring and heating. Once a clear solution is obtained, Crodafos™ CES (10 g), White Petrolatum (10 g), Isopropyl Palmitate (5.0 g), Methylparaben (0.2 g) and Propylparaben (0.05 g) are added gradually under stirring and the mixture is allowed to cool to room temperature. Purified Water q.s. ad 100 (47.45 g) is then added gradually while stirring for 30 more minutes to obtain 100 g of LUT-017 0.03% w/w cream.

### Example 2

### LUT-014 (Formula (I), R=3-(trifluoromethoxy)phenyl) 0.025% w/w Cream Composition

### Composition

### LUT-014 0.025% w/w

### White Petrolatum 10.0% w/w

### Isopropyl Palmitate 5.0% w/w

### Crodafos™ CES 10.0% w/w

Hexylene glycol 2.0% w/w
Diethylene glycol monoethyl ether (Transcutol P™) 25.0% w/w

### Methylparaben 0.2% w/w

### Propylparaben 0.05% w/w

### Purified Water q.s. ad 100 (47.5%)

### Procedure

LUT-014 (0.025 grams), was weighed and charged into a 250 mL three-necked spherical glass flask provided with a mechanical stirrer, a thermometer and a funnel.

Diethylene glycol monoethyl ether (Transcutol P™, 25 g) was added gradually with stirring to the flask containing LUT-014, kept in a water bath heated at 50 deg C. Hexylene glycol (2 g) was then added gradually while stirring and heating. Once a clear solution was obtained, Crodafos™ CES (10g), White Petrolatum (10 g), Isopropyl Palmitate (5 g), Methylparaben (0.2 g) and Propylparaben (0.05 g) was added gradually under stirring and the mixture was allowed to cool to room temperature. Purified Water q.s. ad 100 (47.5 g) was then added gradually while stirring for 30 more minutes to obtain 100 g of LUT-014 0.025% w/w cream.

### Example 3

LUT-017 (Formula (I), R=4-chloro-3-(trifluoromethyl)phenyl) 0.03% w/w Becaplermin 0.01% w/w Combination Cream Composition

### Composition

### LUT-017 0.03% w/w

### Becaplermin 0.01% w/w

### White Petrolatum 10.0% w/w

### Isopropyl Palmitate 5.0% w/w

### Crodafos™ CES 10.0% w/w

Hexylene glycol 2.0% w/w
Diethylene glycol monoethyl ether (Transcutol PTM™) 25.0% w/w

### Methylparaben 0.2% w/w

### Propylparaben 0.05% w/w

### Purified Water q.s. ad 100 (47.45%)

### Procedure

LUT-017 (0.03 g), is weighed and charged into a 250 mL three-necked spherical glass flask provided with a mechanical stirrer, a thermometer and a funnel.

Diethylene glycol monoethyl ether (Transcutol PTM, 25 g) is added gradually with stirring to the flask containing LUT-017, kept in a water bath heated at 50 deg C. Once a clear solution is obtained, it is allowed to cool to room temperature. In a separate vessel, becaplermin (0.01 g) is dissolved in hexylene glycol (2 g) gradually while stirring at room temperature and the obtained becaplermin solution is added to the LUT-017 solution while stirring, Crodafos™ CES (10 g), White Petrolatum (10 g), Isopropyl Palmitate (5.0 g), Methylparaben (0.2 g) and Propylparaben (0.05 g) are added gradually under stirring. Purified Water q.s. ad 100 (47.45 g) is then added gradually while stirring for 30 more minutes to obtain 100 g of LUT-017 0.03% w/w/becaplermin 0.01% w/w cream.

The combination cream must be stored refrigerated at 2°-8° C. (36°-46° F.) and is not to be freezed.

### Example 4

Wound Treatment with LUT-017 (Formula (I), R=4-chloro-3-(trifluoromethyl)phenyl) 0.03% w/w Cream

A wound is prepared for treatment by cleansing and, if needed, debriding. The wound is treated with LUT-017 0.03% w/w/cream once daily, by topically applying the cream (with or without a wound dressing or bandage) on the wound in an amount commensurate with the wound size.

After the treatment, the wound is healed.

### Example 5

Wound Treatment with LUT-017 (Formula (I), R=4-chloro-3-(trifluoromethyl)phenyl) 0.03% w/w and Becaplermin 0.01% w/w Combination

### Cream Composition

A wound is prepared for treatment by cleansing and, if needed, debriding.

The wound is treated with LUT-017 0.03% w/w /becaplermin 0.01% w/w combination cream once daily, by topically applying the cream (with or without a wound dressing or bandage) on the wound in an amount commensurate with the wound size. After the treatment, the wound is healed

### Example 6

Wound Treatment with LUT-017 (Formula (I), R=4-chloro-3-(trifluoromethyl)phenyl) 0.03% w/w Cream on Alternate Days in Combination with Regranex (Becaplermin) Gel 0.01% w/w

A wound is prepared for treatment by cleansing and, if needed, debriding.

The wound is treated with LUT-017 0.03% w/w/cream and Regranex 0.01% gel once daily on alternate days according to the Regranex dosage and administration instructions and LUT-017 method of treatment detailed in Example 3.

After the treatment, the wound is healed

### Example 7

LUT-013 (Formula (I), R =3-(1,1-Dimethylethyl)-1-methyl-1H-pyrazol-5-yl) 0.03% w/w Cream Composition

### Composition

### LUT-013 0.03% w/w

### White Petrolatum 10.0% w/w

### Isopropyl Palmitate 5.0% w/w

### Crodafos™ CES 10.0% w/w

Hexylene glycol 2.0% w/w
Diethylene glycol monoethyl ether (Transcutol PTM) 25.0% w/w

### Methylparaben 0.2% w/w

### Propylparaben 0.05% w/w

### Purified Water q.s. ad 100 (47.45%)

### Procedure

LUT-013 (0.03 grams), is weighed and charged into a 250 mL three-necked spherical glass flask provided with a mechanical stirrer, a thermometer and a funnel.

Diethylene glycol monoethyl ether (Transcutol PTM, 25 g) is added gradually with stirring to the flask containing LUT-017, kept in a water bath heated at 50 deg C. Hexylene glycol (2 g) is then added gradually while stirring and heating. Once a clear solution is obtained, Crodafos™ CES (10 g), White Petrolatum (10 g), Isopropyl Palmitate (5.0 g), Methylparaben (0.2 g) and Propylparaben (0.05 g) are added gradually under stirring and the mixture is allowed to cool to room temperature. Purified Water q.s. ad 100 (47.45 g) is then added gradually while stirring for 30 more minutes to obtain 100 g of LUT-013 0.03% w/w cream.

All publications, patents mentioned herein are hereby incorporated by reference in their entirety as if each individual publication or patent was specifically and individually indicated to be incorporated by reference.

What is claimed is:

1. A pharmaceutical composition for treatment, healing and/or ameliorating a wound in a subject in need thereof comprising a therapeutically effective amount of a compound of Formula (I) or combinations thereof, optionally a therapeutically effective amount of an additional therapeutic agent, and a pharmaceutically acceptable carrier or excipient, 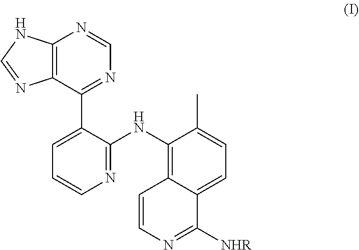
wherein R is 3-chloro-4-fluorophenyl, 2-fluoro-4-iodophenyl, 4-chloro-3-(trifluoromethyl)phenyl, 3 -(1,1-dimethylethyl)-1 -methyl-1H-pyrazol-5-yl, 3-(trifluoromethoxy)phenyl, or a pharmaceutical acceptable salt or solvate thereof,
wherein the optional additional therapeutic agent comprises a pro-angiogenic agent, a growth factor, a sugar, an antacid, vitamin A, vitamin D, an antimicrobial, an antiseptic, an analgesic, a matrix metalloproteinase, a delta-like ligand 4, class 3 Semaphorin or combinations thereof.

2. The pharmaceutical composition of claim 1, wherein in the compound of Formula (I), R is 4-chloro-3-(trifluoromethyl)phenyl.

3. The pharmaceutical composition of claim 1, wherein in the compound of Formula (I), R is 3-(trifluoromethoxy)phenyl.

4. The pharmaceutical composition of claim 1, wherein in the compound of Formula (I), R is 3-(1,1-dimethylethyl)-1-methyl-1H-pyrazol-5-yl.

5. The pharmaceutical composition of claim 1, wherein the growth factor comprises is a fibroblast growth factor, a vascular endothelial growth factor (VEGF), a platelet-derived growth factor (PDGF), a placental growth factor (PIGF), an angiopoietin, or a combination thereof.

6. The pharmaceutical composition of claim 1, wherein the additional therapeutic agent is becaplermin or aCT1 peptide.

7. The pharmaceutical composition of claim 1, wherein the composition is formulated for topical administration.

8. The pharmaceutical composition of claim 1, wherein the composition is in the form of a gel, a hydrogel, an ointment, a cream, a foam, a spray, a lotion, a liquid or a dermal patch.

9. The pharmaceutical composition of claim 1, wherein the compound of Formula (I) is present at a concentration of 0.01% w/w to 2% w/w of the total weight of the composition. 20 10. The pharmaceutical composition of claim 1, wherein the compound of Formula (I) is present at a concentration of 2% w/w to 5% w/w of the total weight of the composition.

11. A method of treatment, healing and/or ameliorating a wound in a subject in need thereof, comprising administering to the subject in need thereof a pharmaceutical composition comprising a therapeutically effective amount of a compound of Formula (I) or combinations thereof and optionally a therapeutically effective amount of an additional therapeutic agent, and a pharmaceutically acceptable carrier or excipient, 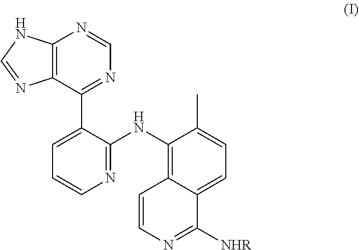
wherein R is 3-chloro-4-fluorophenyl, 2-fluoro-4-iodophenyl, 4-chloro-3-(trifluoromethyl)phenyl, 3-(1,1-dimethylethyl)-1-methyl-1H-pyrazol-5-yl, 3-(trifluoromethoxy)phenyl, or a pharmaceutical acceptable salt or solvate thereof,
wherein a sufficient amount of the pharmaceutical composition increases activity of Mitogen-Activated Protein Kinase by at least 2 times compared to activity of Mitogen-Activated Protein Kinase of a composition comprising a compound of Formula (I) wherein R is p-chlorophenyl, and
wherein the sufficient amount of the pharmaceutical composition is effective for treatment, healing, and/or amelioration of a wound.

12. The method of claim 11, wherein in the compound of Formula (I), R is 4-chloro-3-(trifluoromethyl)phenyl.

13. The method of claim 11, wherein in the compound of Formula (I), R is 3-(trifluoromethoxy)phenyl.

14. The method of claim 11, wherein in the compound of Formula (I), R is 3-(1,1-dimethylethyl)-1-methyl-1H-pyrazol-5-yl.

15. The method of claim 11, wherein the wound is a non-penetrating wound selected from abrasions, lacerations and contusions, a penetrating wound selected from stab wounds, superficial cuts, scratches or lacerations, post-operative wounds, surgical incisions and wounds and gunshot wounds, a thermal wound selected from burns, sunburns and frostbite, an ulcer selected from chronic diabetic ulcers and pressure ulcers/bedsores, a chemical wound, animal or insect bites and stings and electrical wounds.

16. The method of claim 11, wherein the wound is caused by a disorder, and wherein the disorder is Epidermolysis Bullosa (EB), Stevens-Johnson Syndrome (SJS), Toxic Epidermal Necrolysis (TEN), Staphylococcal Scaled Skin Syndrome (SSSS). Pemphigus vulgaris (PV), or Toxic Shock Syndrome (TSS).

17. The method of claim 11, comprising administering topically the pharmaceutical composition to the subject in need thereof.

18. The method of claim 11, comprising administering topically the pharmaceutical composition to the subject in need thereof, wherein the pharmaceutical composition is in the form of a gel, a hydrogel, an ointment, a cream, a spray, a wound dressing, a dermal patch, a foam, a lotion or a liquid.

19. Use of a compound of Formula (I) or combinations thereof 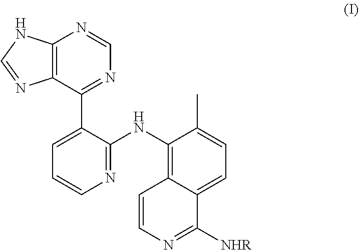
wherein R is 3-chloro-4-fluorophenyl, 2-fluoro-4-iodophenyl, 4-chloro-3-(trifluoromethyl)phenyl, 3-(1,1-dimethylethyl)-1-methyl-1H-pyrazol-5-yl, 3-(trifluoromethoxy)phenyl, or a pharmaceutical acceptable salt or solvate thereof, and optionally an additional therapeutic agent, for preparation of a medicament for treatment, healing and/or ameliorating a wound in a subject in need thereof.

20. Use of pharmaceutical composition comprising a therapeutically effective amount of a compound of Formula (I) or combinations thereof, optionally a therapeutically effective amount of an additional therapeutic agent, and a pharmaceutically acceptable carrier or excipient, 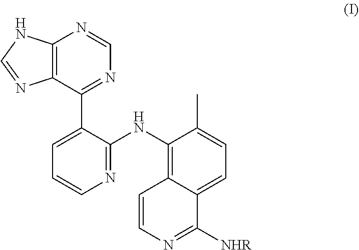
wherein R is 3-chloro-4-fluorophenyl, 2-fluoro-4-iodophenyl, 4-chloro-3-(trifluoromethyl)phenyl, 3-(1,1-dimethylethyl)-1-methyl-1H-pyrazol-5-yl, 3-(trifluoromethoxy)phenyl, or a pharmaceutical acceptable salt or solvate thereof,
wherein a sufficient amount of the pharmaceutical composition increases activity of Mitogen-Activated Protein Kinase by at least 2 times compared to activity of Mitogen-Activated Protein Kinase of a composition comprising a compound of Formula (I) wherein R is p-chlorophenyl, and
wherein a sufficient amount of the pharmaceutical composition is effective for treatment, healing, and/or amelioration of a wound.
